(19) United States
(12) Patent Application Publication
(10) Pub. No.: US 2008/0108967 A1
(43) Pub. Date: May 8, 2008

(54) Disposable Pull-On Absorbent Article Comprising an Outergarment-Like Graphic

(75) Inventors: Tomoe Mizushima, Kobe (JP); Mai Nakamura, Setagaya-ku (JP)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(73) Assignee: The Procter & Gamble Company

(21) Appl. No.: 11/935,453

(22) Filed: Nov. 6, 2007

Related U.S. Application Data
(60) Provisional application No. 60/857,044, filed on Nov. 6, 2006.

(51) Int. Cl. A61F 13/15; A61B 17/06

(52) U.S. Cl. 604/385.23; 206/438; 604/385.01

(57) ABSTRACT

A disposable pull-on absorbent article comprises an outergarment-like graphic. The outergarment-like graphic provides a design theme and comprises an outergarment component element and a background element. The outergarment component element and the background element are selected to be interrelated in terms of the design theme. The design theme is characterized by a combination of the outergarment component element and the background element.

The present invention also relates to an array of the disposable pull-on absorbent articles contained in a package. Each of the absorbent articles comprises an outergarment-like graphic, which provides a design theme and comprises an outergarment component element and a background element, and the outergarment component element and the background element are selected to be interrelated in terms of the design theme. The design theme is characterized by a combination of the outergarment component element and the background element.

### CROSS REFERENCE TO RELATED APPLICATION

This application claims the benefit of U.S. Provisional Application No. 60/857,044, filed Nov. 6, 2006, the substance of which is incorporated herein by reference.

### FIELD OF THE INVENTION

The present invention relates to a disposable pull-on absorbent article comprising an outergarment-like graphic. The present invention further relates to an array of disposable pull-on absorbent articles contained in a package, each absorbent article comprising said outergarment-like graphic.

### BACKGROUND OF THE INVENTION

Diapers are absorbent articles which absorb and contain urine and other body exudates. Diapers for toddlers can be categorized into two types in terms of structure. One is a “conventional diaper” comprising closure means so as to form the diaper into an essentially closed configuration around the wearer's body (e.g. taped diaper). The other is a “pull-on diaper” with closed side seams. Pull-on diapers are designed mainly for the purpose of being worn by toddlers who can stand or walk by themselves.

In the growing process of toddlers, the toddlers gradually learn to recognize the outer “visual stimulation” of objects such as colors, sizes, shapes, patterns, graphics and the like. The diapers which toddlers wear are not an exception. Toddlers may think that such diapers whose design seems to be made just for small infants are dull.

The manufacturers have paid attention mainly to the functions of the diapers such as liquid absorption ability, comfortableness, breathability and the like. In contrast, the manufacturers may not be keen to provide a variety of designs in diapers. The design of a diaper mainly comprises a traditional plain or unpatterned background. However, such a traditional design may not sufficiently appeal to toddlers, parents or caregivers visually when the toddlers wear diapers or when diapers are subject to exchange. Toddlers who are ready to graduate from conventional diapers may want another concept of design in what they wear, not a traditional concept of design.

Some diapers adopt a character design on the outer surface in the front and/or in the back of the diapers to attract toddlers. However, the diapers with, for example, a character design alone still may give an impression to toddlers that the diapers are designed for young infants. Furthermore, diapers with a character design alone may not include a fashion element. Therefore, there has been no choice of fashion-oriented diapers for toddlers.

Recently, there have been various kinds of children's garments. It may be partly because the parents' generation is familiar with various kinds of garments which they themselves wore. In raising their children, parents may tend to reflect their preference for garments on their children's garments.

Based on the foregoing, it may be desirable to provide an improved fashion element. It may be also desirable to provide pleasure in the choice of a diaper from a package which contains a plurality of diapers.

### SUMMARY OF THE INVENTION

The present invention relates to a disposable absorbent article comprising an outergarment-like graphic. The outergarment-like graphic provides a design theme and comprises an outergarment component element and a background element. The outergarment component element and the background element are selected to be interrelated in terms of a design theme. The design theme is characterized by a combination of the outergarment component element and the background element.

The present invention also relates to an array of a plurality of the above described disposable pull-on absorbent articles contained in a package. The array of disposable absorbent articles comprises a first absorbent article comprising a first outergarment-like graphic and a second absorbent article comprising a second outergarment-like graphic. The first outergarment-like graphic provides a first design theme and the second outergarment-like graphic provides a second design theme. The first outergarment-like graphic and the second outergarment-like graphic are different from each other by differentiating at least one selected from the outergarment component element and the background element between the first outergarment-like graphic and the second outergarment-like graphic. The first design theme and the second design theme may be relevant to each other. Alternatively, the first design theme and the second design theme may be irrelevant to each other.

The above-mentioned and other features and advantages of the present invention and the manner of attaining them will become more apparent, and the invention itself will be better understood by reference to the drawings and the following description of the drawings.

### BRIEF DESCRIPTION OF THE DRAWINGS

While the specification concludes with claims which particularly point out and distinctly claim the invention, it is believed that the present invention will be better understood from the following description of embodiments taken in conjunction with the accompanying drawings, in which like reference numerals identify identical elements and wherein:

FIG. 1 is a front plan view of an embodiment of the present invention;

FIG. 2 is a back plan view of the embodiment depicted in FIG. 1;

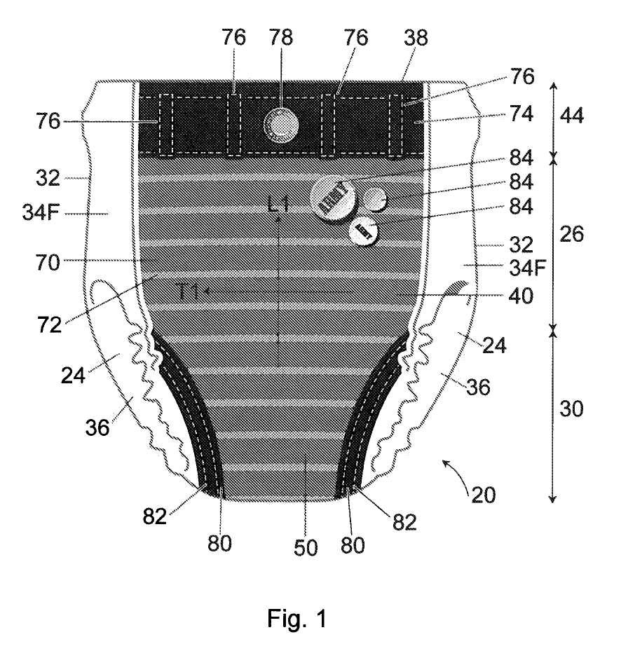

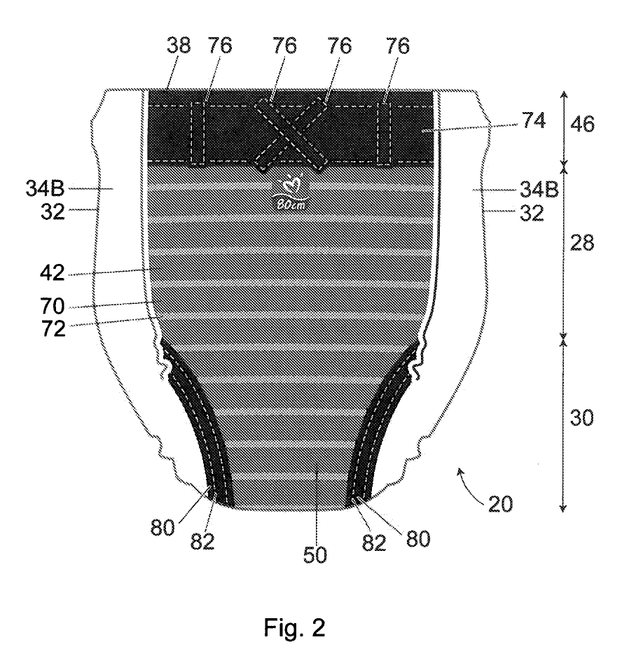

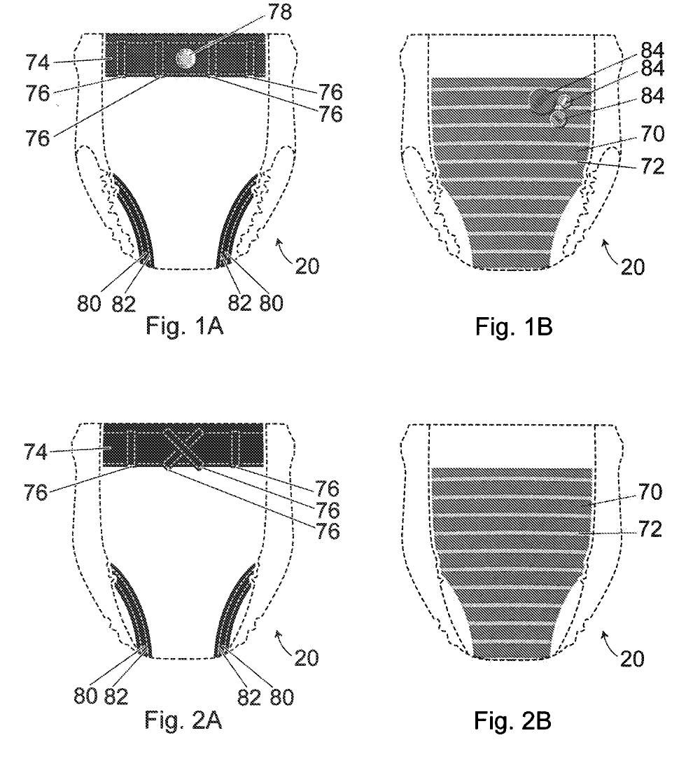

FIG. 1A is a partial view showing the outergarment component element of the outergarment-like graphic of the embodiment depicted in FIG. 1;

FIG. 1B is a partial view showing the background element of the outergarment-like graphic of the embodiment depicted in FIG. 1;

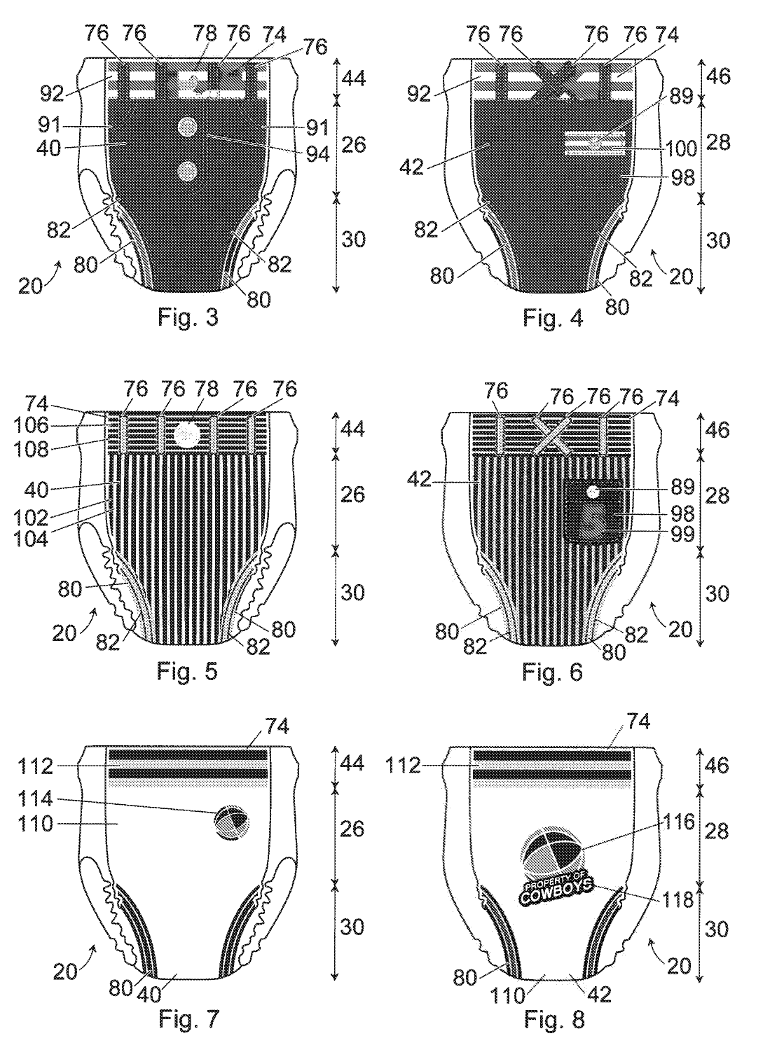

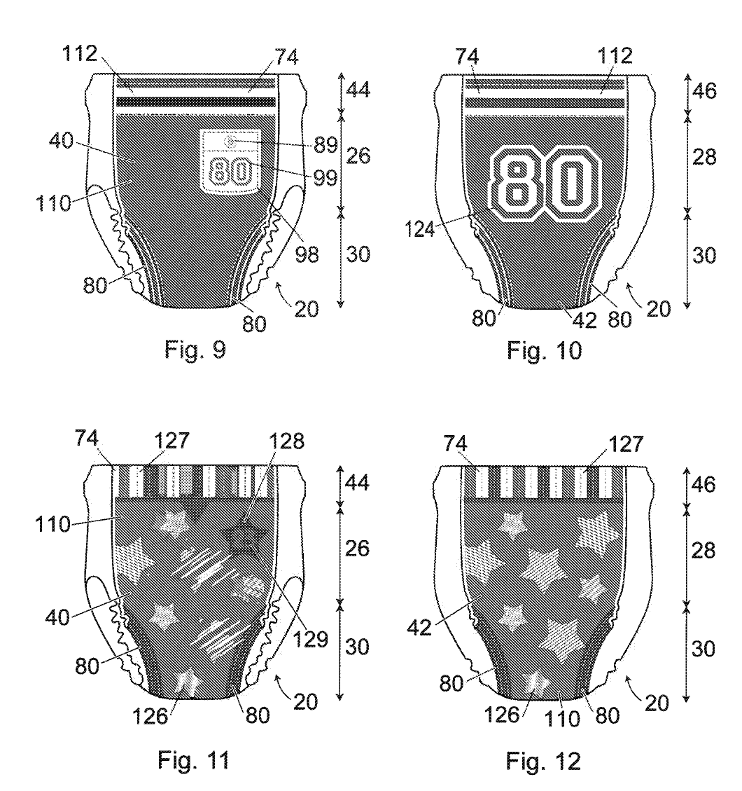

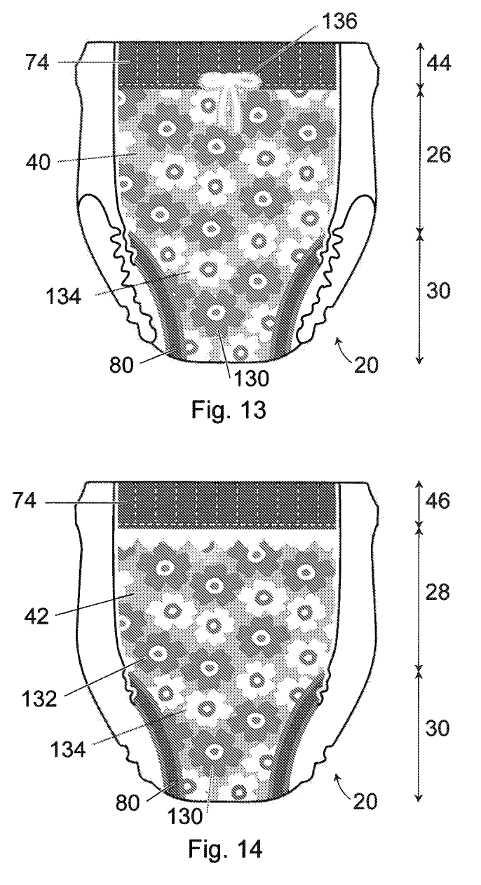

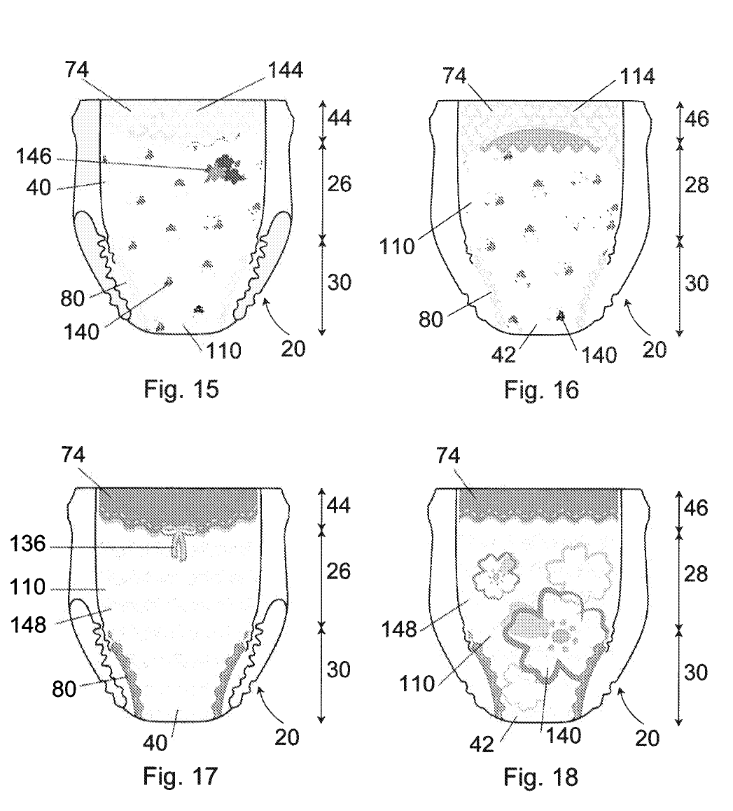

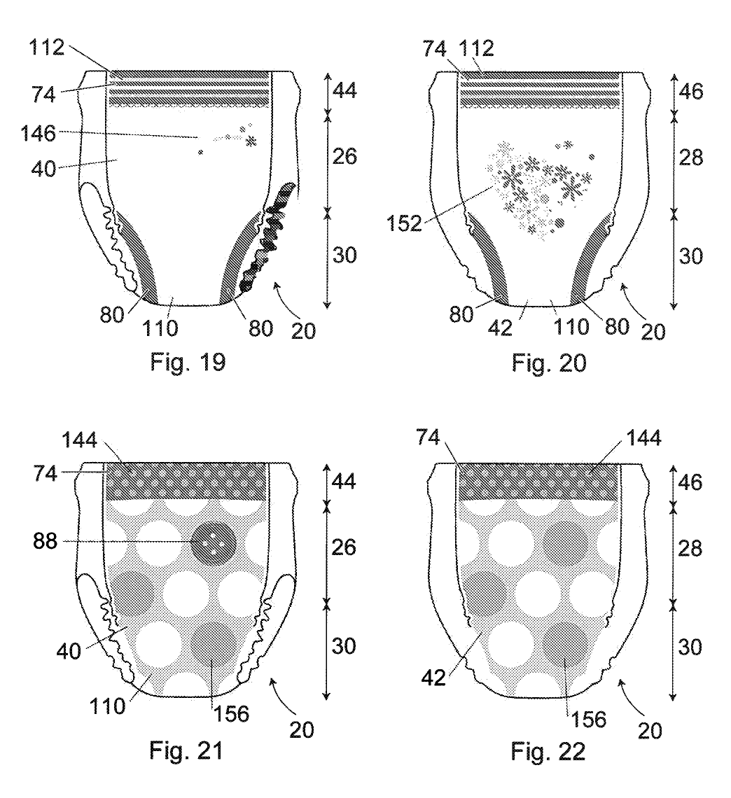

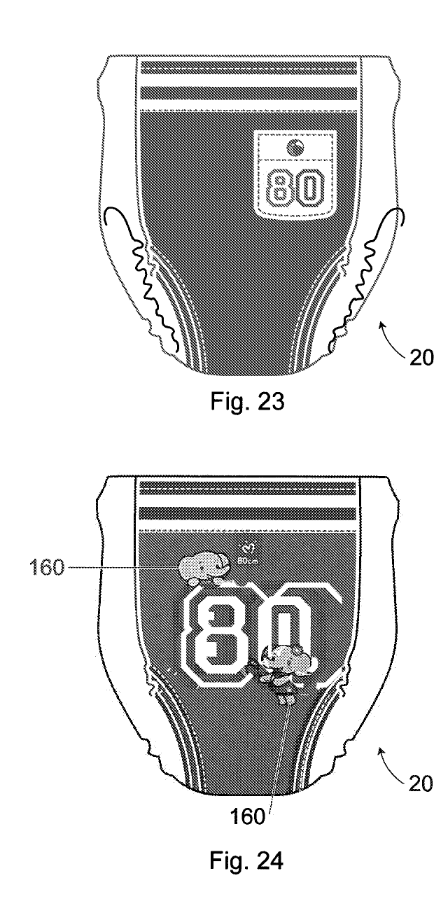

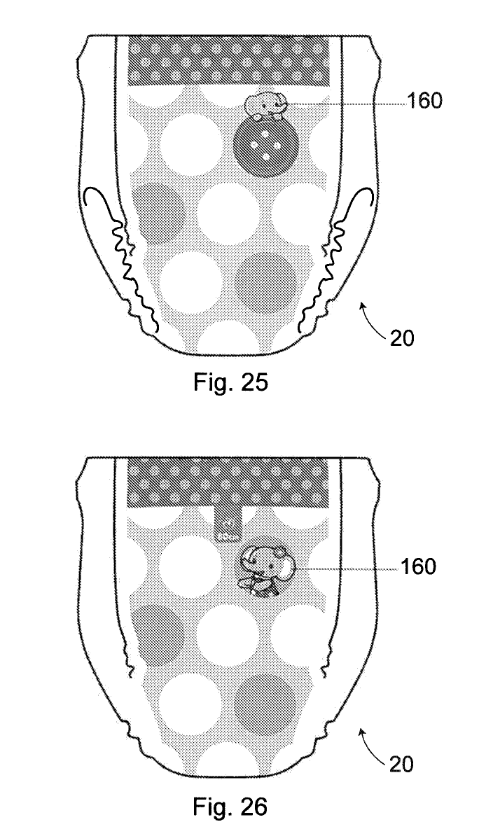

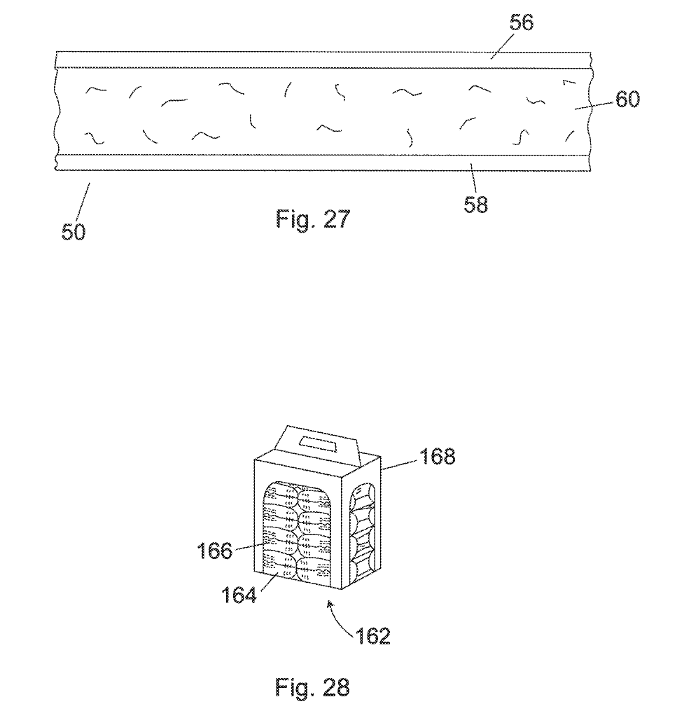

FIG. 2A is a partial view showing the outergarment component element of the outergarment-like graphic of the embodiment depicted in FIG. 2;

FIG. 2B is a partial view showing the background element of the outergarment-like graphic of the embodiment depicted in FIG. 2;

FIG. 3 is a front plan view of another embodiment of the present invention;

FIG. 4 is a back plan view of the embodiment depicted in FIG. 3;

FIG. 5 is a front plan view of another embodiment of the present invention;

FIG. 6 is a back plan view of the embodiment depicted in FIG. 5;

FIG. 7 is a front plan view of another embodiment of the present invention;

FIG. 8 is a back plan view of the embodiment depicted in FIG. 7;

FIG. 9 is a front plan view of another embodiment of the present invention;

FIG. 10 is a back plan view of the embodiment depicted in FIG. 9;

FIG. 11 is a front plan view of another embodiment of the present invention;

FIG. 12 is a back plan view of the embodiment depicted in FIG. 11;

FIG. 13 is a front plan view of another embodiment of the present invention;

FIG. 14 is a back plan view of the embodiment depicted in FIG. 13;

FIG. 15 is a front plan view of another embodiment of the present invention;

FIG. 16 is a back plan view of the embodiment depicted in FIG. 15;

FIG. 17 is a front plan view of another embodiment of the present invention;

FIG. 18 is a back plan view of the embodiment depicted in FIG. 17;

FIG. 19 is a front plan view of another embodiment of the present invention;

FIG. 20 is a back plan view of the embodiment depicted in FIG. 19;

FIG. 21 is a front plan view of another embodiment of the present invention;

FIG. 22 is a back plan view of the embodiment depicted in FIG. 21;

FIG. 23 is a front plan view of another embodiment of the present invention;

FIG. 24 is a back plan view of the embodiment depicted in FIG. 23;

FIG. 25 is a front plan view of another embodiment of the present invention;

FIG. 26 is a back plan view of the embodiment depicted in FIG. 25;

FIG. 27 is a partial section view of the main body of the embodiment of the present invention; and

FIG. 28 is a perspective schematic view of an array of absorbent articles in a package.

In the Figures, lead lines on the colored background are used for visual convenience and do not mean that the Figures have such outlined lines actually.

### DETAILED DESCRIPTION OF THE INVENTION

The present invention relates to a disposable pull-on absorbent article. The absorbent article comprises an outergarment-like graphic. The outergarment-like graphic provides a design theme to the absorbent article.

The term “absorbent article” means a diaper, training pants, an incontinence product, diaper pants, disposable underwear or the like. Suitable training pants and diaper pants can have seamed side proportions or refastenable side portions. The present invention may be suited for use with training pants or diapers for toddlers.

The term “disposable” means an article which is not intended to be laundered or otherwise restored or reused (i.e., they are intended to be discarded after a single use and preferably, to be recycled, composted or otherwise disposed of in an environmentally compatible manner). The term “pull-on article” means an article of wear which has a defined waist opening and a pair of leg openings and which are pulled onto the body of the wearer by inserting the legs into the leg openings and pulling the article up over the waist. The “pull-on article” may be also “absorbent” to absorb and contain urine and other body exudates. One embodiment of the present invention may be a pull-on diaper such as a diaper 20, for example, shown in FIGS. 1 and 2. The “pull-on diaper” is generally worn by toddlers and other incontinent individuals to absorb and contain urine and other body exudates. It should be understood, however, that the present invention is also applicable to other pull-on absorbent articles such as training pants, incontinence briefs, feminine hygiene garments or panties and the like.

The term “outergarment” means clothing which is seen from outside when worn on the body, and sometimes worn over other garments like undershirts or underpants. In the present invention, the term “outergarment” includes pants, slacks, jeans, trousers, dresses (e.g., one-piece dresses, two-piece dresses, party dresses), shirts (e.g., sweat shirts, T-shirts, tanktop, polo shirts, dress shirts), blouses, breeches, scarves, shawls, overalls, camisoles, vests, sweaters, skirts, culottes, tank tops, jackets, sport wears (e.g., soccer wears, swimming wears, golf wears, tennis wears, rugby wears, basketball wears, training wears, jerseys) and the like, but it is not limited thereto. The term “outergarment-like” means that an absorbent article whose outer appearance simulates an outergarment. The term “graphic” means a design, a drawing or a picture which is used to attract persons who see the absorbent article such as parents, caregivers and toddlers (hereinafter referred to as “viewers”).

The outergarment-like graphic provides a design theme. The term “design theme” means a concept which gives a particular impression to viewers. The concept may reflect a fashion, a trend, a current, a culture, a civilization and the like. Examples of the “design theme” are, “American casual”, “French chic”, “psychedelic”, “Italian casual”, “ethnic”, “marine look”, “Japanesque” and the like.

The design theme of “American casual” means a fashion style which may be explained by the words such as “bright”, “active”, “casual”, “cheerful”, “energetic”, “lively”, “positive”, “magnificent”, “grand”, “powerful”, “strong”, “vital”, “dynamic”, “vigorous” and the like. Its source is a style which emphasizes a fashion classified into some categories such as sports, work, outdoor, military, biker and the like. Also, “American casual” may look better on denim fabric such as jeans. The examples are provided with the FIGS. 1 to 12 described hereafter. In order to provide the design theme of “American casual”, it is beneficial, for example, to provide with a large logo, a large graphic, a clear-cut pattern and the like. And for example, a combination of a dark color and a bright color to give color contrast may provide a design theme of “American casual” because the design theme may be explained by the words such as “active” and “vigorous.”

The design theme of “French chic” means a fashion style which may be explained by the words such as “elegant”, “graceful”, “tender”, “calm”, “quiet”, “decent”, “peaceful”, “mild”, “static”, “serene”, “smart”, “chaste” and the like. Its source is a style which mainly comprises pastel and faint colors such as light yellow, faint pink, light blue, light green, white, gray, beige, ivory and the like. The examples are provided with FIGS. 13 to 22 described hereafter. In order to provide the design theme of “French chic”, it is beneficial, for example, to provide a light color and a feminine fashion motif such as a flower, a leaf, grass, a ribbon, a frill, a lace, a polka dot, a wavy line and the like, and fine and minute patterns or objects, subtle color gradations and the like because the design theme may be explained by the words such as “elegant” and “graceful.”

The design theme of “psychedelic” means a fashion style comprising outstanding gaudy colors, fluorescent colors and the like, as if one could experience when taking a psychedelic drug.

The design theme of “Italian casual” means a fashion style which may be explained by the words such as “modern”, “adult”, “sophisticated”, “refined” and the like. “Italian casual” may sometimes reflect a fashion design. “Italian casual” may comprise dim colors but it gives an impression of “modern” because of the high fashion sense.

The design theme of “ethnic” means a fashion style of a country or culture that is different from western culture and therefore novel and interesting for western people. “Ethnic” is also called “folklore” which provides a fashion taste of racial traditions. In particular, “ethnic” is meant to specify Middle and Near East, Africa, Middle and South America, central Asia, Mongol and the like. Some ethnic fashions have a taste of traditional flowers, plants, trees, leaves, animals, reptiles, insects and the like.

The design theme of “marine look” imitates clothing which sailors wear. Examples for the clothing include, sailor collars, sailor hats, white culottes. Also, the clothing comprising a motif such as a yacht, an anchor, a ship, a boat, a helm may be included. “Marine look” may be also called “navy look.”

The design theme of “Japanesque” means a fashion style of an ethnic taste, especially relating to a traditional Japanese culture. “Japanesque” means an old Japanese fashion taste, for example, exemplified by sumi-e (Japanese ink brush painting), a kimono (literally “something one wears”, but in this context it especially relates to a Japanese traditional garment), a sumo wrestler, yukata (informal unlined summer kimono usually made of cotton, linen or hemp), cherry blossoms, Hiragana or Katakana (also called Japanese characters), Kanji (also called Chinese characters) and the like.

The outergarment-like graphic comprises an outergarment component element and a background element and the outergarment-like graphic provides a design theme to the diaper. The “design themes” may be different by differentiating at least one of the outergarment component element or the background element.

The “design theme” may be characterized by a combination of the outergarment component element and the background element. For example, when the background element is plain and the plain background element gives nothing or little (e.g., a white plain pattern) to the design theme, the outergarment component element more strongly contributes to a design theme than the background element. In such a case, the background element may not interfere with providing the design theme, but the background element may reinforce the outergarment component element to characterize a specific design theme. Also, when the outergarment component element provides nothing or little to the design theme, the background element may more strongly contribute to a design theme than the outergarment component element. In such a case, the outergarment component element may not interfere with providing the design theme, but the outergarment component element may reinforce the background element to provide a specific design theme. The combination of the outergarment component element and the background element may also provide a design theme.

The term “outergarment component element” means an element of the absorbent article which imitates a component in a particular section of the outergarment that has a specific function in the outergarment. The “outergarment component element” includes, for example, a simulated waistband, a simulated belt, a simulated belt hole, a simulated pocket, a simulated button, a simulated leg band, a simulated stitch, a simulated zip, a simulated pants fly, a simulated tag for design theme, a size mark and the like. More than one of the outergarment component elements may be combined together.

The term “background element” means an element of the absorbent article which occupies the majority of the whole absorbent article. Examples of the background element include a plain monotone, regular or irregular arrangement of lines or shapes and the like as a design on material. The “background element” includes, for example, a plain color, a stripe, a check, a plaid, a line (e.g. a straight line, wavy line, a polygonal line), a drawing (e.g., a polka dot, an astral object, a flower drawing, a leaf drawing, a ball drawing (a basketball, a baseball, a volleyball, a tennis ball and the like), a bat drawing), a tartan, a gingham check, a herringbone, a hound's-tooth, a millefleur, an overcheck, a waffle, a shepherd's plaid, a Greek key, an argyle, a checker, a Prince of Wales check, a lozenge, a rectangle, a teardrop, a square and the like. More than one of the background elements may be joined together.

The background element may further include an “ornament element.” The term “ornament element” means an object which further contributes to the design theme by decorating the background element. The “ornament element” may include, for example, a letter (e.g., comprising alphabet, a Chinese character, a numeral), a logo, a numeral, a mark, a simulated badge, a simulated emblem, a simulated frill, a simulated ribbon and the like. More than one of the ornament elements may be joined together.

The term “interrelated” means that the selection of an outergarment component element and a background element is carried out in order to exert a synergetic effect to provide a specific design theme. The specific design theme is not limited particularly, but examples of the specific design theme include “American casual”, “French chic”, “psychedelic”, “Italian casual”, “ethnic”, “marine look”, “Japanesque” and the like. For example, when the outergarment component element provides a design theme of “American casual”, the selection of the background element may be carried out to provide a design theme of “American casual” to exert a synergetic effect to provide a common design theme of “American casual.” Also, when the background element provides a design theme of “American casual”, the selection of the outergarment component element may be carried out to provide a design theme of “American casual” to exert a synergetic effect to provide a common design theme of “American casual.” The combination of the outergarment component element and the background element may provide a common design theme.

The following is a detailed explanation of the absorbent article in the present invention, exemplified in FIGS. 1 and 2.

The pull-on diaper 20 illustrated in FIGS. 1 and 2 has a longitudinal centerline L1 and a transverse centerline T1. The diaper 20 comprises a main body 50 comprising a liquid permeable topsheet 56, a backsheet 58 and an absorbent core 60 therebetween (see FIG. 27). The liquid permeable topsheet 56 may be positioned adjacent to the body-facing surface of the absorbent core 60 and may be joined thereto and to the backsheet 58 by any suitable attachment means. The backsheet 58 is generally that portion of the diaper 20 positioned adjacent to the external surface of the absorbent core 60 and prevents the urine and other body exudates absorbed and contained in the absorbent core 60 from soiling articles that may contact the diaper 20. The absorbent core 60 between the liquid permeable topsheet 56 and the backsheet 58 absorbs and retains the urine and other body exudates. The liquid permeable topsheet 56, the backsheet 58 and the absorbent core 60 may be manufactured from any suitable materials. Examples for the material of the backsheet 58 include, a film and the like, and the backsheet may be covered with further materials such as nonwoven. The main body 50 may additionally comprise a barrier leg cuff disposed along the longitudinal side edge. The barrier leg cuff may provide improved containment of liquids and other body exudates in the crotch region 30.

The diaper 20 has an outer surface 22, an inner surface 24 opposed to the outer surface 22. The diaper 20 comprises a main body 50, a front side panel 34F and a back side panel 34B. The main body 50 comprises a front region 26, a back region 28 and a crotch region 30. The front side panel 34F and the back side panel 34B extend transversely outwardly from the main body 50. The front side panel 34F and the back side panel 34B are joined at the seams 32 in the right and in the left respectively. The front side panels 34F and the back side panels 34B join the front region 26 and the back region 28 to form a pair of leg openings 36 and a waist opening 38. The front region 26 comprises a front main panel 40 and a front waist panel 44. The back region 28 comprises a back main panel 42 and a back waist panel 46.

The front waist panel 44 comprises a portion of the diaper 20 positioned in the front of the wearer. The back waist panel 46 comprises a portion of the diaper 20 positioned in the back of the wearer. The crotch region 30 comprises a portion of the diaper 20 between the legs of the wearer and covers the lower torso of the wearer.

The front side panel 34F and the back side panel 34B (collectively referred to as side panels 34) comprise an outer cover layer, an inner layer, and an elastic intermediate layer therebetween. The outer cover layer may comprise any known materials. The outer cover layer comprises a single layer of nonwoven. The outer cover layer may comprise a single layer of hydrophobic nonwoven. The inner layer may comprise any suitable materials. The inner layer may comprise a single layer of nonwoven. The barrier leg cuffs may extend from the longitudinal side edge transversely outwardly to constitute an outer surface. The outer cover layer may be directly joined to and covers the backsheet 58 of the main body 50.

The outergarment-like graphic of the disposable pull-on absorbent article may be printed on the backsheet 58 such as a film. The film may be covered with nonwoven through which the outergarment-like graphic is visible from outside. The outergarment-like graphic may be printed on the nonwoven. The nonwoven may have a supplement pattern related to the design theme. The term “supplement” means that the pattern appearing on the nonwoven contributes to the design theme synergetically with the outergarment-like graphic of the backsheet 58. When the nonwoven is printed with the outergarment-like graphic, the outer surface 22 of the backsheet 58 may not be printed with the outergarment-like graphic.

The outergarment-like graphic in the diaper 20 may also be arranged to appear on a part of the side panels 34, continuously or discontinuously from the graphic on the front main panel 40 and/or the back main panel 42. At least one of the side panels 34 may have a side pattern related to the design theme of the front main panel 40 and/or the back main panel 42. The term “related” means that the side pattern of the side panels 34 provides the same design theme as that of the outergarment-like graphic on the front main panel 40 and/or back main panel 42 or supplements the design theme. Otherwise, the outergarment-like graphic which provides a design theme may be printed only on the side panels 34.

FIGS. 1 and 2 illustrate an embodiment of a diaper 20 in which an outergarment-like graphic provides a design theme of “American casual.” The outergarment-like graphic comprises an outergarment component and a background component. The outergarment component element comprises a navy blue simulated waistband 74 and navy blue simulated belt holes 76 in the front waist panel 44 and in the back waist panel 46. The outergarment component element also comprises a simulated button 78 in the center of the front waist panel 44. The outergarment component element further comprises a pair of navy blue simulated leg bands 80 along the edge of the crotch regions 30. The simulated leg bands 80 are highlighted by simulated stitches 82. The simulated stitches 82 contribute to the design theme on the pair of simulated leg bands 80. The background element comprises an ornament element of badges 84. The background element comprises horizontal stripes in the front main panel 40 and in the back main panel 42. The horizontal stripes comprise light blue thick lines 70 and gray thin lines 72 disposed alternately. The thickness, colors and the number of the stripes are not limited thereto. For example, the stripes may comprise a plurality of stripes having the same thickness. The shape of the stripes is not limited to horizontal stripes, but vertical stripes, diagonal stripes, a combination of stripes in different directions may be included as well.

The outergarment-like graphic comprised in the diaper 20 illustrated in FIGS. 1 and 2 provides a design theme of “American casual.” The horizontal stripes, the simulated waistband 74, the simulated leg bands 80, the simulated button 78, and the simulated badges 84 are selected to be interrelated in terms of a design theme of “American casual.” The combination of the outergarment component element (the simulated waistband 74, the simulated leg bands 80 and a simulated button 78; see FIGS. 1A and 2A) and the background element (horizontal stripes and simulated badges 84; see FIGS. 1B and 2B) provides a design theme of “American casual.” Also, the outergarment-like graphic comprised in the diaper 20 illustrated in FIGS. 1 and 2 provides a design theme of “American casual” by imitating “sweat shirt.” The “sweat shirt” is worn by boys and girls, and particularly worn as casual clothing and it provides an impression explained by “casual”, “lively” and the like. Therefore, the “sweat shirt” may lead to a design theme of “American casual.”

The present invention may provide an effect of giving pleasure to toddlers, parents and caregivers of choosing an absorbent article comprising an outergarment-like graphic which provides a design theme of “American casual.” The absorbent article looks like a garment, and therefore it may attract toddlers, parents and caregivers without wearing actual skirts or pants. For example, praising toddlers like “cute”, “cool”, “lovely” has an effect on toddlers' growth similar to the phrases such as “Well done!”, “Good for you!”, “I am proud of you!” and the like. It may help toddlers get confident. The absorbent article may affect the mental growth of toddlers. When toddlers are ready to graduate from conventional diapers, both parents and toddlers may want to realize that the toddlers are steadily growing. The absorbent article of the present invention can help parents and toddlers realize the growth of the toddlers. Since the graphics appearing on the surface of an absorbent article imitate outergarment, toddlers are willing to wear them. The absorbent article may promote toddlers to phase from conventional diapers to pull-on diapers (such as training pants) smoothly by its design theme. A plurality of said absorbent articles gives toddlers, parents and caregivers pleasure when the absorbent articles are subject to exchange. As described hereinbefore, the absorbent articles adopting the design of outergarment may promote toddlers' mental growth.

The absorbent article of the present invention may further comprise a character. The character may include an animal such as an elephant, a boy, a girl, a toy, a dog, a cat, a giraffe, a sheep, a wolf, a horse, a mouse, a duck, a bear, a dolphin, a fish, a tiger, a lion and the like. The character can be an animation character such appears in Sesame Street™ and the like.

The outer surface 22 of the main body 50 may be filled with the outergarment-like graphic. The outergarment-like graphic may be arranged to appear on at least about 70% or at least about 80% of the area of the main body 50.

FIGS. 3 and 4 illustrate another embodiment of a diaper 20 in which an outergarment-like graphic provides a design theme of “American casual.” The outergarment-like graphic comprises an outergarment component element and a background element. The outergarment component element comprises a simulated waistband 74 of red and white and simulated belt holes 76 in the front waist panel 44 and in the back waist panel 46. The outergarment component element also comprises a pair of simulated leg bands 80 along the ends of the crotch region 30. The simulated leg bands 80 are highlighted by simulated stitches 82. The simulated leg bands 80 comprise red lines and white lines. The outergarment component element further comprises simulated buttons 78 in the center of the front waist panel 44 and in the front main panel 40 and a pair of simulated pockets 91 in the front main panel 40. Furthermore, the outergarment component element comprises a simulated pants fly 94 and a simulated pocket 98 provided with a simulated flap 100. The simulated flap is provided with a simulated button 89 in the center. The background element comprises a simulated “denim fabric” in the front main panel 40 and in the back main panel 42. The pattern of the “denim fabric” is plain navy blue.

The simulated waistband 74 comprises horizontal stripes 92. The horizontal stripes 92 comprise red, white and dark brown parts. The simulated buttons 78 are aligned along the longitudinal centerline L1. One of the simulated buttons (first button) is positioned in the center of the front waist panel 44 and two buttons are aligned along the longitudinal centerline L1 below the first button in the front main panel 40. The “denim fabric” simulated belt holes 76 highlighted by simulated stitches 82 are positioned over the simulated waistband 74. The simulated pockets 91 are positioned in the front main panel 40 and they are highlighted by simulated stitches 82. The simulated pants fly 94 is highlighted by simulated stitches 82 beside the align of the simulated buttons 78. The simulated flap 100 is characterized under the same design theme of “American casual” as the simulated waistband 74. The background element further comprises an ornament element of a simulated button 89 on the simulated pocket 98 in the back main panel 42. The two simulated belt holes 76 are positioned to form an X-mark in the center of the back waist panel 46.

The outergarment-like graphic comprised in the diaper 20 illustrated in FIGS. 3 and 4 provides a design theme of “American casual.” The simulated denim fabric, the simulated waistband 74, the simulated buttons 78, the simulated leg bands 80, the simulated pants fly 94 and the simulated pocket 98 are selected to be interrelated in terms of a design theme of “American casual.” The combination of the outergarment component element (the simulated waistband 74, the simulated leg bands 80 and a simulated button 78) and the background element (horizontal stripes and simulated badges 84) provides a design theme of “American casual.” Also, the outergarment-like graphic comprised in the diaper 20 illustrated in FIGS. 3 and 4 provides a design theme of “American casual” by imitating “denim jeans.” The “denim jeans” are worn as a labor wear or a casual wear. The “denim jeans” provide an impression of “conspicuous”, “outstanding”, “bold”, “active” and the like. The “denim jeans” may lead to a design theme of “American casual.” A combination of a solid color and a bright color next to each other (i.e. color contrast) provides a design theme of “American casual.” A combination of a plurality of colors may also be acceptable. The arrangement of the colors is not limited thereto, but an alignment of colors of a solid color next to a bright color can be preferable for expressing a design theme of “American casual.” The colors, numbers, and the way of arrangement of the buttons are not limited to the example, but any modification may be acceptable as far as they provide a design theme of “American casual.” For example, black, gray, light blue or any color may be acceptable as far as it provides a design theme of “American casual.” The simulated buttons 78 are a symbol for denim fabric. The red lines 96 of the crotch regions 30 provide a design theme in contrast with navy blue of the denim fabric.

FIGS. 5 and 6 illustrate another embodiment of a diaper 20 in which an outergarment-like graphic provides a design theme of “American casual.” The outergarment-like graphic comprises an outergarment component element and a background element. The outergarment component element comprises a simulated waistband 74 in the front waist panel 44 and in the back waist panel 46. The simulated waistband 74 comprises horizontal stripes 112. The horizontal stripes 112 comprise navy blue parts 106 and white parts 108 disposed alternately. The outergarment component element comprises a pair of gray simulated leg bands 80 along the ends of crotch region 30. The outergarment component element comprises simulated gray belt holes 76 in the front waist panel 44. The outergarment component element comprises a simulated button 78 in the center of the front waist panel 44. The outergarment component element also comprises a simulated pocket 98 in the back main panel 42. The simulated pocket 98 comprises a logo of numeral “5” 99 and a simulated button 89 inside. The simulated pocket is highlighted by simulated stitches 82. The two gray simulated belt holes 76 are positioned to form an X-mark in the center of the back waist panel 46. The background element comprises vertical stripes in the front main panel 40 and in the back main panel 42. The vertical stripes comprise navy blue lines 102 and white lines 104 disposed alternately in the front region 26 and in the back region 28. The colors are not limited thereto, but any combination which has sharp contrast may be acceptable.

The outergarment-like graphic comprised in the diaper 20 illustrated in FIGS. 5 and 6 provides a design theme of “American casual.” The vertical stripes, the simulated waistband 74 and the simulated leg bands 80 are selected to be interrelated in terms of a design theme of “American casual.” The combination of the outergarment component element (the simulated waistband 74 and the simulated leg bands 80) and the background element (the vertical stripes and the simulated pocket 98) characterizes the design theme of “American casual.” The logo 99 further contributes to characterize the design theme of “American casual.” Also, the outergarment-like graphic comprised in the diaper 20 illustrated in FIGS. 5 and 6 provides a design theme of “American casual” by imitating an “overall.” The “overall” is worn by children, preferably by boys and it provides an impression of “bold”, “cheerful” and the like. The “overall” may lead to a design theme of “American casual.”

FIGS. 7 and 8 illustrate another embodiment of a diaper 20 in which an outergarment-like graphic provides a design theme of “American casual.” The outergarment-like graphic comprises an outergarment component element and a background element. The outergarment component element comprises a simulated waistband 74 in the front waist panel 44 and in the back waist panel 46. The simulated waistband 74 comprises horizontal stripes 112. The horizontal stripes 112 comprise red, gray and navy blue lines. Each line is provided almost in the same width, but the widths may vary to each other. The outergarment component element comprises a pair of simulated leg bands 80 along the ends of the crotch region 30. The simulated leg bands 80 comprise navy blue lines, red lines and white lines disposed alternately. The outergarment component element comprises a drawing of basketball 116 in the center. An outlined logo “PROPERTY OF COWBOYS” 118 overlaps the basketball drawing 116. The background element comprises a white ground 110 in the front main panel 40 and in the back main panel 42. The background element also comprises an ornament element of a basketball logo 114 whose colors are consistent with the horizontal stripes 112.

The outergarment-like graphic comprised in the diaper 20 illustrated in FIGS. 7 and 8 provides a design theme of “American casual.” The basketball drawings 114 and 116, the simulated waistband 74 and the simulated leg bands 80 are selected to be interrelated in terms of a design theme of “American casual.” The combination of the outergarment component element (the simulated waistband 74 and the simulated leg bands 80) and the background element (the petal drawings 132 and 134 and the simulated ribbon 136) provides a design theme of “American casual.” In particular, the arrangement of the stripes in the simulated waistband 74 provides a design theme of “American casual.” Also, the outergarment-like graphic comprised in the diaper 20 illustrated in FIGS. 7 and 8 provides a design theme of “American casual” by imitating a “T-shirt.” The “T-shirt” is worn as a sport wear or as a casual wear, and it provides an impression of “active”, “energetic” and the like. The “T-shirt” may lead to a design theme of “American casual.”

FIGS. 9 and 10 illustrate another embodiment of a diaper 20 in which an outergarment-like graphic provides a design theme of “American casual.” The outergarment-like graphic comprises an outergarment component element and a background element. The outergarment component element comprises a simulated waistband 74 in the front waist panel 44 and in the back waist panel 46, and a pair of red simulated leg bands 80 along the ends of the crotch regions 30. The simulated waistband 74 comprises horizontal stripes 112. The horizontal stripes comprise red, white and dark brown lines. The color combination of the simulated leg bands 80 is consistent with the simulated waistband 74. The outergarment component element further comprises a white simulated pocket 98 with a logo of numeral “80” 99 of outlined red characters and with a simulated button 89 inside. The background element comprises a brick red plain ground 110 in the front main panel 40 and in the back main panel 42. The background element also comprises an ornament element of an outlined red logo of numeral “80” 124 in the back main panel 42.

The outergarment-like graphic comprised in the diaper 20 illustrated in FIGS. 9 and 10 provides a design theme of “American casual.” The simulated waistband 74, the simulated leg bands 80 and the simulated pocket 98 are selected to be interrelated in terms of a design theme of “American casual.” The combination of the outergarment component element (the simulated waistband 74 and the simulated leg bands 80) and the background element (the ground 40 and the simulated pocket 98) provides a design theme of “American casual.” Also, the outergarment-like graphic comprised in the diaper 20 illustrated in FIGS. 9 and 10 provides a design theme of “American casual” by imitating a “sport wear.” The “sport wear” provides an impression of “active”, “cheerful”, “energetic”, “vital” and the like. The “sport wear” may lead to a design theme of “American casual.”

FIGS. 11 and 12 illustrate another embodiment of a diaper 20 in which an outergarment-like graphic provides a design theme of “American casual.” The outergarment component element comprises a simulated waistband 74 in the front waist panel 44 and in the back waist panel 46. The simulated waistband 74 comprises vertical stripes 127 consisting of red, white and blue parts. The background element comprises a blue ground 110 and light blue astral objects 126 are scattered over the ground 110 in the front main panel 40 and in the back main panel 42. The background element also comprises a deep blue astral object 128 in the front main panel 40. The background further element comprises an ornament element of a numeral logo of “23” 129 in the center of the deep blue astral object 128.

The outergarment-like graphic comprised in the diaper 20 illustrated in FIGS. 11 and 12 provides a design theme of “American casual.” The astral objects 126, the simulated waistband 74 and the simulated leg bands 80 are selected to be interrelated in terms of a design theme of “American casual.” The combination of the outergarment component element (the simulated waistband 74 and the simulated leg bands 80) and the background element (the astral objects 126 and 128, and the numeral logo 129) provides a design theme of “American casual.” Also, the outergarment-like graphic comprised in the diaper 20 illustrated in FIGS. 11 and 12 provides a design theme of “American casual” by imitating a T-shirt and adopting decoration of “astral objects.” The “T-shirt” and “astral objects” with the sharp contrast with the background provide an impression of “active”, “energetic”, “magnificent” and the like. They may lead to a design theme of “American casual.”

The diapers illustrated in FIGS. 1 to 12 comprise outergarment-like graphics which provide design themes of “American casual.” For example, the diaper illustrated in FIGS. 1 and 2 simulates a sweat shirt. The diaper illustrated in FIGS. 3 and 4 simulates denim jeans. The diaper illustrated in FIGS. 5 and 6 simulates an overall. The diaper illustrated in FIGS. 7 and 8 simulates a T-shirt. The diaper illustrated in FIGS. 9 and 10 simulates a sport wear. The diaper illustrated in FIGS. 11 and 12 simulates a T-shirt. All of them may remind viewers of a design theme of “American casual.”

FIGS. 13 and 14 illustrate an embodiment of a diaper 20 in which an outergarment-like graphic provides a design theme of “French chic.” The outergarment component element comprises a pink simulated waistband 74 in the front waist panel 44 and in the back waist panel 46. The outergarment component element also comprises a pair of pink simulated leg bands 80 along the ends of the crotch regions 30. The background element comprises flower drawings 130 in the front main panel 40 and in the back main panel 42. The flower drawings comprise some faint pink petal drawings 132 and some white petal drawings 134. The background element also comprises an ornament element of a simulated ribbon 136 hung over the center of the pink simulated waistband 74 in the front main panel 40.

The outergarment-like graphic comprised in the diaper 20 illustrated in FIGS. 13 and 14 provides a design theme of “French chic.” The petal drawings 132 and 134, the simulated waistband 74 and the simulated leg bands 80 are selected to be interrelated in terms of a design theme of “French chic.” The combination of the outergarment component element (the simulated waistband 74 and the simulated leg bands 80) and the background element (the petal drawings 132 and 134, and the simulated ribbon 136) provides a design theme of “French chic.” Also, the outergarment-like graphic comprised in the diaper 20 illustrated in FIGS. 13 and 14 provides a design theme of “French chic” by imitating a “girl's one-piece dress” and adopting decoration of flower petals. The “girl's one-piece dress” and “flower petals” provide an impression of “elegant”, “mild” and the like, and it may lead to a design theme of “French chic.”

FIGS. 15 and 16 illustrate another embodiment of a diaper 20 in which an outergarment-like graphic provides a design theme of “French chic.” The outergarment-like graphic comprises an outergarment component element and a background element. The outergarment component element comprises a faint pink simulated waistband 74 in the front waist panel 44 and in the back waist panel 46 and white polka dots 144 are placed regularly in the simulated waistband 74. A bouquet drawing 146 of several kinds of flowers are positioned in the front main panel 40. The outergarment component element also comprises a pair of faint pink simulated leg bands 80 along the ends of the crotch region 30. The background element comprises a white ground 110 and flower drawings 140 scattered on the white ground 110 in the front main panel 40 and in the back main panel 42.

The outergarment-like graphic comprised in the diaper 20 illustrated in FIGS. 15 and 16 provides a design theme of “French chic.” The white ground 110, the flower drawings 140, the bouquet drawing 146, the simulated waistband 74, and the simulated leg bands 80 are selected to be interrelated in terms of a design theme of “French chic.” The combination of the outergarment component element (the simulated waistband 74 and the simulated leg bands 80) and the background element (the white ground 110, the flower drawings 140 and the bouquet drawing 146) provides a design theme of “French chic.” Also, the outergarment-like graphic comprised in the diaper 20 illustrated in FIGS. 15 and 16 provides a design theme of “French chic” by imitating a “girl's blouse” and adopting decoration of flowers. The “girl's blouse” and “flowers” provide an impression of “elegant”, “decent” and the like, and it may lead to a design theme of “French chic.”

FIGS. 17 and 18 illustrate another embodiment of a diaper 20 in which an outergarment-like graphic provides a design theme of “French chic.” The outergarment-like graphic comprises an outergarment component element and a background element. The outergarment component element comprises a light blue simulated waistband 74 in the front waist panel 44 and in the back waist panel 46, in which the lower end is wavy in the front main panel 40 and in the back main panel 42. The outergarment component element comprises a pair of simulated leg bands 80 along the ends of the crotch region 30. The background element comprises some wavy lines 148 in a white ground 110 in the front main panel 40 and in the back main panel 42. The background element also comprises some large flower drawings 140 in the back main panel 42. The background element further comprises an ornament element of a simulated ribbon 136 hung in the lower center of the frill.

The outergarment-like graphic comprised in the diaper 20 illustrated in FIGS. 17 and 18 provides a design theme of “French chic.” The white ground 110, the wavy lines 148, the simulated ribbon 136, the flower drawings 140, the simulated waistband 74, and the simulated leg bands 80 are selected to be interrelated in terms of a design theme of “French chic.” The combination of the outergarment component element (the simulated waistband 74, and the simulated leg bands 80) and the background element (the white ground 110, the wavy lines 148, the simulated ribbon 136 and flower drawings 140) provides a design theme of “French chic.” Also, the outergarment-like graphic comprised in the diaper 20 illustrated in FIGS. 17 and 18 provides a design theme of “French chic” by imitating a “girl's one-piece dress” and adopting decoration of large flowers. The “girl's one-piece dress” and “large flowers” also provide an impression of “elegant”, “graceful” and the like, and it may lead to a design theme of “French chic.”

FIGS. 19 and 20 illustrate another embodiment of a diaper 20 in which an outergarment-like graphic provides a design theme of “French chic.” The outergarment-like graphic comprises an outergarment component element and a background element. The outergarment component element comprises a simulated waistband 74 consisting of horizontal stripes 112 in the front waist panel 44 and in the back waist panel 46. The horizontal stripes comprise pink, orange and white lines. Also, the outergarment component element comprises a pair of pink simulated leg bands 80 along the ends of the crotch region 30. The background element comprises a white ground 110 in the front main panel 40 and in the back main panel 42. The background element also comprises a bouquet drawing 146 consisting of several flowers in the front main panel 40. The background element further comprises an ornament element consisting of a heart-shaped drawing 152 filled with flower drawings in the center of the back main panel 42.

The outergarment-like graphic comprised in the diaper 20 illustrated in FIGS. 19 and 20 provides a design theme of “French chic.” The bouquet drawing 146, a heart-shaped drawing 152, the simulated waistband 74 and the simulated leg bands 80 are selected to be interrelated in terms of a design theme of “French chic.” The combination of the outergarment component element (the simulated waistband 74 and the simulated leg bands 80) and the background element (the bouquet drawing 146, a heart-shaped drawing 152 and the ground) provides a design theme of “French chic.” Also, the outergarment-like graphic comprised in the diaper 20 illustrated in FIGS. 19 and 20 provides a design theme of “French chic” by imitating a “blouse” and adopting decoration of a bouquet and a heart-shaped flower drawing. The “girl's blouse” and the “flower drawing” also provide an impression of “elegant”, “graceful” and the like, and it may lead to a design theme of “French chic.”

FIGS. 21 and 22 illustrate another embodiment of a diaper 20 in which an outergarment-like graphic provides a design theme of “French chic.” The outergarment-like graphic comprises an outergarment component element and a background element. The outergarment component element comprises a blue simulated waistband 74 in the front waist panel 44 and in the back waist panel 46. The simulated waistband 74 is filled with light blue small polka dots 144. The background element comprises a plain light blue ground 110 in the front main panel 40 and in the back main panel 42. The background element also comprises polka-dots 156 consisting of white and light blue. The background element also comprises an ornament element of a polka dot imitating a button 88 in the front main panel 40.

The outergarment-like graphic comprised in the diaper 20 illustrated in FIGS. 21 and 22 provides a design theme of “French chic.” The polka dots 156, the ground 110 and the simulated waistband 74 are selected to be interrelated in terms of a design theme of “French chic.” The combination of the outergarment component element (the simulated waistband 74) and the background element (the ground 110 and the polka dots 156) provides a design theme of “French chic.” Also, the outergarment-like graphic comprised in the diaper 20 illustrated in FIGS. 21 and 22 provides a design theme of “French chic” by imitating “a girl's skirt” and adopting decoration of polka dots. The “girl's one-piece dress” and the “polka dots” also provide an impression of “serene”, “tender”, “calm” and the like, and it may lead to a design theme of “French chic.”

The diapers of FIGS. 13 to 22 comprise outergarment-like graphics which provide the design theme of “French chic.” For example, the diaper illustrated in FIGS. 13 and 14 simulates a girl's one-piece dress. The diaper illustrated in FIGS. 15 and 16 simulates a girl's blouse. The diaper illustrated in FIGS. 17 and 18 simulates girl's one-piece dress. The diaper illustrated in FIGS. 19 and 20 simulates a blouse. The diaper illustrated in FIGS. 21 and 22 simulates a girl's skirt. All of them remind the viewers of a design theme of “French chic.”

FIGS. 23 and 24 show an embodiment of a pull-on diaper 20 of the present invention, comprising an elephant character of “PAMPA™” 160 in addition to an outergarment-like graphic which provides a design theme of “American casual” based on the diaper illustrated in FIGS. 9 and 10. In this embodiment, the front main panel (FIG. 23) does not comprise an elephant character, but it may also comprise it.

FIGS. 25 and 26 show an embodiment of a pull-on diaper 20 of the present invention, comprising an elephant character of “PAMPA™” 160 in addition to an outergarment-like graphic which provides a design theme of “French chic” based on the diaper illustrated in FIGS. 21 and 22. For example, the “PAMPA™” design has been used widely. As illustrated in FIGS. 23 to 26, the combination of the outergarment-like graphic and the “PAMPA™” character provides a friendly impression to toddlers.

The present invention further relates to an array of a plurality of the absorbent articles as described hereinbefore contained in a package. The term “array” relates to a plurality of disposable pull-on absorbent articles contained in a package. The array may be an array of diapers or training pants. The term “package” means, for example, a resin package, a paper package and the like. Each absorbent article comprises an outergarment-like graphic and the outergarment-like graphic provides a design theme and comprises an outergarment component element and a background element. The outergarment component element and the background element are selected to be interrelated in terms of the design theme. The design theme is characterized by the combination of the outergarment component element and the background element. The array of the disposable absorbent articles comprises a first absorbent article comprising a first outergarment-like graphic and a second absorbent article comprising a second outergarment-like graphic. The first outergarment-like graphic provides a first design theme, and the second outergarment-like graphic provides a second design theme. The first outergarment-like graphic and the second outergarment-like graphic are different from each other by differentiating at least one selected from the outergarment component element and the background element (including ornament element) between the first outergarment-like graphic and the second outergarment-like graphic. When the outergarment component elements of the first and the second outergarment-like graphics are different from each other and the background elements of them are the same, the design themes may be the same or different from each other. When the outergarment component elements of the first and the second outergarment-like graphics are the same and the background elements of the first and the second outergarment-like graphics are different from each other, the design themes may be the same or different from each other. When both the outergarment component elements and the background elements are different from each other, the design themes may be the same or different from each other. When the design theme of the first outergarment-like graphic and that of the second outergarment-like graphic are the same, the first design theme and the second design theme are relevant. When the design theme of the first outergarment-like graphic and that of the second outergarment-like graphic are different, the first design theme and the second design theme are irrelevant.

The term “differentiating” means that the first graphic and the second graphic have a different outergarment component element, background element and/or a combination thereof. The design of the diaper is not limited to the above outergarment component element and background element, but any combination will be acceptable as far as the outergarment-like graphic provides a specific design theme.

The term “relevant” means that the first design theme and the second design theme are identical to each other (e.g., the first design theme is “American casual”, and the second design theme is “American casual”), regardless of differentiating at least one selected from the outergarment component element and the background element between the first outergarment-like graphic and the second outergarment-like graphic.

The term “irrelevant” means that the first design theme and the second design theme are not identical to each other (e.g., the first design theme is “American casual” and the second design theme is “French chic”, because “American casual” and “French chic” have greatly different design themes from each other) by differentiating at least one selected from the outergarment component element and the background element between the first outergarment-like graphic and the second outergarment-like graphic.

FIG. 28 illustrates a schematic perspective view of an array 162 of a plurality of diapers 164 each comprising an outergarment-like graphic (not shown in FIG. 28) in a package. The outergarment-like graphic provides a design theme and comprises an outergarment component element and a background element. The outergarment component element and the background element are selected to be interrelated in terms of the design theme. The design theme is characterized by the outergarment component element, the background element and/or the combination thereof.

The array 162 in FIG. 28 comprises a first diaper 164 comprising a first outergarment-like graphic and a second diaper 166 comprising a second outergarment-like graphic. “The first diaper” means any one absorbent article arbitrarily selected from the array consisting of two absorbent articles or more. “The second diaper” means any one absorbent article arbitrarily selected from the array one diaper has been drawn from the above array. The first outergarment-like graphic in the first diaper 164 provides a first design theme and the second outergarment-like graphic in the second diaper 166 provides a second design theme. The array 162 is contained in a package 168.

The first outergarment-like graphic and the second outergarment-like graphic are different from each other by differentiating at least one selected from the outergarment component element and the background element between the first outergarment-like graphic and the second outergarment-like graphic, and the first design theme and the second design theme may be relevant or irrelevant to each other. The purpose of differentiation is to give pleasure to toddlers by selecting and unwrapping the packed diapers.

FIG. 28 discloses an array consisting of four diapers, but the number of the diaper is not limited thereto. The number of the diaper is preferably 2 to 50, more preferably 2 to 30.

The outergarment-like graphic comprised in the diapers which provide a design theme will attract toddlers, parents and caregivers visually to assist the growth of toddlers from the point of view of mentality, urination and excretion.

The absorbent main body 50 absorbs and contains urine and other body exudates disposed on the main body 50. In the embodiment shown in FIG. 1, the main body 50 has a generally rectangular shape having a longitudinal centerline L1, a transverse centerline T1, left and right longitudinally extending side edges and front and back transversely extending end edges.

The dimensions and values disclosed herein are not to be understood as being strictly limited to the exact numeral values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A disposable pull-on absorbent article comprising an outergarment-like graphic,
the outergarment-like graphic providing a design theme and comprising an outergarment component element and a background element,
said outergarment component element and said background element being selected to be interrelated in terms of a design theme, and
said design theme being characterized by a combination of said outergarment component element and said background element.

2. The disposable pull-on absorbent article of claim 1 wherein said outergarment component element comprises a simulated waistband, a simulated belt, a simulated belt hole, a simulated pocket, a simulated button, a simulated leg band, a simulated stitch, a simulated zip, a simulated pants fly, a tag for design theme, a size mark or any combination thereof.

3. The disposable pull-on absorbent article of claim 1 wherein the background element comprises an ornament element comprising a letter, a logo, a numeral, a mark, a simulated badge, a simulated emblem, a simulated frill, a simulated ribbon, a character or any combination thereof.

4. The disposable pull-on absorbent article of claim 1 wherein said absorbent article extends in a longitudinal direction and in a transverse direction, said absorbent article comprising a main body comprising a liquid permeable topsheet, a backsheet, and an absorbent core therebetween, and a pair of side panels extending transversely outwardly from the main body, wherein said outergarment-like graphic is arranged to appear on at least 70% of the area of the main body.

5. The disposable pull-on absorbent article of claim 4 wherein said outergarment-like graphic is arranged to appear on a part of the side panel.

6. The disposable pull-on absorbent article of claim 4 wherein said outergarment-like graphic is printed on said backsheet, and said backsheet is covered with nonwoven.

7. The disposable pull-on absorbent article of claim 6 wherein said nonwoven has a supplement pattern related to the design theme.

8. The disposable pull-on absorbent article of claim 4 wherein said side panel has a side pattern related to said design theme.

9. An array of a plurality of disposable pull-on absorbent articles contained in a package, each absorbent article comprising an outergarment-like graphic, the outergarment-like graphic providing a design theme and comprising an outergarment component element and a background element, the outergarment component element and the background element being selected to be interrelated in terms of the design theme, and the design theme being characterized by a combination of the outergarment component element and the background element, wherein
the array of disposable absorbent articles comprises a first absorbent article comprising a first outergarment-like graphic and a second absorbent article comprising a second outergarment-like graphic, the first outergarment-like graphic provides a first design theme, the second outergarment-like graphic provides a second design theme,
the first outergarment-like graphic and the second outergarment-like graphic are different from each other by differentiating at least one selected from the outergarment component element and the background element between the first outergarment-like graphic and the second outergarment-like graphic, and the first design theme and the second design theme are relevant to each other.

10. An array of disposable pull-on absorbent articles contained in a package, each absorbent article comprising an outergarment-like graphic, the outergarment-like graphic providing a design theme and comprising an outergarment component element and a background element, the outergarment component element and the background element being selected to be interrelated in terms of the design theme, and the design theme being characterized by a combination of the outergarment component element and the background element, wherein
the array of disposable absorbent articles comprises a first absorbent article comprising a first outergarment-like graphic and a second absorbent article comprising a second outergarment-like graphic, the first outergarment-like graphic provides a first design theme, the second outergarment-like graphic provides a second design theme,
the first outergarment-like graphic and the second outer-garment-like graphic are different from each other by differentiating at least one selected from the outergarment component element and the background element between the first outergarment-like graphic and the second outer-garment-like graphic, and the first design theme and the second design theme are irrelevant to each other.
